(19) United States
(12) Patent Application Publication
(10) Pub. No.: US 2021/0000985 A1
(43) Pub. Date: Jan. 7, 2021

(54) Gel for Acoustic Coupler, Method for Producing the Same, and Ultrasonic Imaging Method

(71) Applicant: Hitachi, Ltd., Tokyo (JP)

(72) Inventors: Kenichi KAWABATA, Tokyo (JP); Hideki YOSHIKAWA, Tokyo (JP)

(21) Appl. No.: 16/898,047

(22) Filed: Jun. 10, 2020

(30) Foreign Application Priority Data
Jul. 5, 2019 (JP) 2019-126250

(51) Int. Cl. A61K 49/22; A61B 8/00; C08F 222/38; C08K 5/1545

(52) U.S. Cl. CPC A61K 49/226; C08K 5/1545; C08F 222/385; A61B 8/4281

(57) ABSTRACT

To provide an acoustic coupler capable of achieving both acoustic characteristics and mechanical characteristics required for ultrasonic imaging. The invention provides a hydrogel preparation method in which a plurality of kinds of polymerizations are performed under reduced pressure, and a gel for an acoustic coupler obtained by the preparation method. For example, a gel for an acoustic coupler includes polyacrylamide having a matrix structure and alginic acid, and the alginic acid is retained in a matrix of the matrix structure of the polyacrylamide.

### TECHNICAL FIELD

The present invention relates to a coupler that performs acoustic coupling between an ultrasonic transmission and reception probe and an irradiation target, and the coupler is used for an apparatus that performs imaging based on a signal obtained by irradiating a body with an ultrasonic wave.

### BACKGROUND ART

In modern medicine, image diagnosis capable of non-invasively obtaining information inside body is an indispensable technique and is widely used. In particular, there is great expectation on solutions enabled by a small and inexpensive ultrasonic diagnostic apparatus among image diagnostic modalities.

In other modalities such as X-ray CT or MRI, a subject enters an apparatus and a whole body is imaged, whereas in an ultrasonic diagnostic apparatus, a probe is pressed against a part of the subject to be imaged to acquire internal information in real time. Use of such an imaging method has a good aspect that it is possible to just image a region of interest in detail. However, a procedure of an imaging person, such as a degree and angle at which the probe is pressed, is directly reflected on a captured image, and when the imaging person changes, the obtained image also changes, which leads to a problem called “operator dependency”.

One of causes of operator dependency in the ultrasonic diagnostic apparatus is that a method of applying jelly differs slightly depending on the imaging person. A probe of an ultrasonic diagnostic element is pressed against a skin of a subject and an inside of the subject is irradiated with an ultrasonic wave, but body hairs and pores are present on a surface of the skin of the subject, which hinders an input of ultrasonic energy into the living body. For this reason, in order to couple an ultrasonic probe and a living body, an imaging person applies jelly having acoustic impedance close to that of the living body between a probe and a skin, and presses the probe from above the jelly to perform imaging. However, since the jelly cannot keep specific shape, the jelly is spread thinly when the probe is pressed and the probe is almost in contact with the skin. Therefore, it is not easy to cover an irregularity on the surface of the skin with the jelly. In particular, it is difficult to sufficiently fill and smooth the irregularity on the surface with the jelly at a part such as a joint where an irregularity on the surface of the living body is noticeable. As a result, a subtle difference in jelly application caused by an imaging person appears as a noticeable difference in an imaging result.

In addition, in a case of using jelly, when there is a scratch on a surface of a skin of the subject, it is necessary to carefully apply jelly and remove the jelly after inspection, and it is not easy to improve inspection throughputs.

In order to solve such problems of jelly, use of a gel or resin having acoustic impedance close to that of a living body as an acoustic coupler has been proposed in PTL 1 and PTL 2, for example.

### CITATION LIST

### Patent Literature

PTL 1: JP-A-2018-195964

PTL 2: JP-A-2018-175598

### SUMMARY OF INVENTION

### Technical Problem

However, an acoustic coupler formed of a related-art gel or resin is rarely used in a clinical site. A reason is that the related-art gel or resin cannot sufficiently satisfy both acoustic characteristics and mechanical characteristics required for an acoustic coupler in ultrasonic imaging. The acoustic characteristics required for an acoustic coupler are to have acoustic characteristics (sound velocity and attenuation) close to that of a living body (≈water) in order for an ultrasonic wave emitted from a probe to enter the living body. The mechanical characteristics are required such that an acoustic coupler deforms to be brought into close contact with a living body without being destroyed (broken) even when being pressed by a probe. An acoustic coupler known so far satisfies the mechanical characteristics, but an attenuation rate of an ultrasonic wave is high. Therefore, when the acoustic coupler formed of the related-art gel or resin is used, it is difficult to image a deep portion of a living body due to attenuation of an ultrasonic wave, and thus the acoustic coupler formed of the related-art gel or resin is only used in some institutions at a time of imaging a superficial part.

An object of the invention is to provide an acoustic coupler capable of achieving both acoustic characteristics and mechanical characteristics required for ultrasonic imaging.

### Solution to Problem

In order to achieve the above object, the invention provides a gel for an acoustic coupler to be disposed between a subject and a probe that transmits an ultrasonic wave, the gel for acoustic coupler includes: polyacrylamide having a matrix structure and alginic acid, and the alginic acid is formed in the matrix of the matrix structure of the polyacrylamide.

Another aspect of the invention provides a method for producing a gel for an acoustic coupler, the method includes: a step of mixing a plurality of kinds of polymers having different polymerization systems or polymer raw materials; a first gelling step of polymerizing or crosslinking a first kind of polymer or polymer raw material among the plurality of kinds of polymers or the polymer raw materials; and a second gelling step of polymerizing or crosslinking a second kind of polymer or polymer raw material among the plurality of kinds of polymers or the polymer raw materials, and all the steps are performed under reduced pressure.

### Advantageous Effect

The gel for an acoustic coupler produced according to the invention can achieve both acoustic characteristics and mechanical characteristics required for ultrasonic imaging, so that high-quality ultrasonic imaging can be performed regardless of an imaging person.

### BRIEF DESCRIPTION OF DRAWINGS

FIG. 1 shows an example of a mechanical characteristic (breaking strain) measurement result of an acoustic coupler according to an embodiment.

FIG. 2 shows an example of an acoustic characteristic (sound velocity) measurement result of the acoustic coupler according to the embodiment.

FIG. 3 shows an example of an acoustic characteristic (attenuation rate) measurement result of the acoustic coupler according to the embodiment.

FIG. 4 shows an example of a measurement result of variation coefficients of mechanical characteristics and acoustic characteristics of the acoustic coupler according to the embodiment.

FIG. 5 shows an example of a measurement result regarding durability of the acoustic coupler according to the example.

### DESCRIPTION OF EMBODIMENTS

The present inventors have conducted intensive studies and found that a gel for an acoustic coupler capable of achieving both acoustic characteristics and mechanical characteristics required for ultrasonic imaging can be obtained by preparing, under a degassed atmosphere, a composite hydrogel of a hydrogel polymerized using a radical polymerization initiator and a hydrogel formed by polyvalent ion.

For example, a gel for an acoustic coupler can be obtained which includes polyacrylamide having a matrix structure and alginic acid matrix and in which the alginic acid chains are retained in a matrix of the matrix structure of the polyacrylamide. It is desirable that the alginic acid retained in the matrix is crosslinked via an ion to form matrix-shaped alginic acid.

When the gel is disposed between a subject and a probe that transmits an ultrasonic wave, the gel deforms when being pressed by the ultrasonic probe, but the gel is not pushed away like jelly, so that an irregularity on a surface of the subject can be smoothly covered. Moreover, since the acoustic characteristics of the gel are close to that of water, an ultrasonic wave can reach a deep portion without attenuating, and the deep portion can be imaged. Therefore, imaging with reduced operator dependency can be performed.

That is, in a state where the gel (acoustic coupler) according to the present embodiment is disposed between a subject and a probe that transmits an ultrasonic wave, an ultrasonic wave is transmitted from the probe and caused to pass through the acoustic coupler to irradiate an inside of the subject. An ultrasonic wave from the subject toward the probe due to the ultrasonic wave irradiation is caused to pass through the acoustic coupler reach the probe, and to be received. An ultrasonic image is generated using an ultrasonic signal received by the probe. Accordingly, the ultrasonic wave can reach a deep portion with effect of the irregularity on the surface of the subject being prevented and the attenuation of the ultrasonic wave also being controlled, so that an ultrasonic image with reduced operator dependency can be obtained. It is desirable that the gel is disposed such that one surface of the gel is in close contact with a surface of a probe from which an ultrasonic wave is transmitted and the other surface of the gel is in close contact with a body surface of the subject. Therefore, it is possible to form the gel in an appropriate shape in advance in accordance with an imaging part. For example, when a flat body surface such as an abdomen is to be imaged, a gel having a pad (flat plate) shape can be used. When a non-flat three-dimensional (irregular shape) part such as a breast or a joint such as an elbow or a knee is to be imaged, a gel that is formed into a shape that wraps around and flattens the three-dimensional part can be used. Since the gel according to the present embodiment has an attenuation rate equal to that of water, almost no attenuation rate distribution caused by passing through the gel occurs even when a gel with space-wisely different thickness is used, so that imaging can be performed while preventing effect of the irregular shape.

It is desirable that the gel for an acoustic coupler according to the present embodiment has a mechanical characteristic, that is, a strain rate when stretched, of 100% or more, preferably 200% or more, a sound velocity value equal to or less than that of water (within 5% deviation), and further an ultrasonic attenuation rate of 0.1 dB/MHz/cm or less.

As a method for producing the gel for an acoustic coupler, first, a plurality of kinds of polymers (a hydrogel polymerized using a radical polymerization initiator, a hydrogel formed by polyvalent ion, or the like) having different polymerization systems or raw materials thereof are mixed. A first kind of polymer (for example, a hydrogel polymerized using a radical polymerization initiator) is polymerized or crosslinked so as to be gelled. Next, a second kind of polymer (for example, a hydrogel formed by polyvalent ion binding) or a raw material thereof is polymerized or crosslinked so as to be gelled. By performing all these steps under reduced pressure, the gel for an acoustic coupler capable of achieving both acoustic characteristics and mechanical characteristics required for ultrasonic imaging can be produced.

The hydrogel formed by a polymerization using a radical polymerization initiator is preferably polyacrylamide. The hydrogel formed by crosslinking by polyvalent ion is preferably alginic acid crosslinked via a polyvalent ion. As a polyvalent ion source for crosslinking the alginic acid, for example, calcium oxalate can be used. A ratio of the hydrogel polymerized via a radical polymerization initiator to the hydrogel formed by crosslinking by polyvalent ion can be set to 3:2 to 9:1, and preferably 13:7 to 9:1.

The present embodiment is not limited to the above materials. For example, as the hydrogel polymerized using a radical polymerization initiator, diacetone acrylamide, N-hydroxyethyl acrylamide or N-(3-methoxypropyl) acrylamide can be used. As the hydrogel formed by crosslinking by polyvalent ion, LA gellan gum, carrageenan, and LA pectin can be used.

A composition of a desired gel will be clarified by embodiments.

A producing procedure of the present embodiment will be described.

First, a container for holding a polyvalent ion solution and a gel mold for holding a raw material are disposed inside a decompression chamber, and an inside of the decompression chamber is decompressed by a vacuum pump. The polyvalent ion solution, the raw material, and a polymerization agent were introduced into a polyvalent ion server (supply container), a raw material server, and a polymerization agent server, respectively, that are connected to a space in the decompression chamber by transfer pipes, respectively, and then an inside of each server is degassed using an aspirator or the like.

The polyvalent ion solution is transferred from the polyvalent ion server to the polyvalent ion container in the decompression chamber.

Next, the raw material and the polymerization agent are transferred from the raw material server and the polymerization agent server to the gel mold. The gel mold is rotated in an angle range to an extent that the raw material does not spill to mix the raw material and the polymerization agent.

In this state, completion of the radical polymerization of the raw material in the gel mold is awaited.

Next, a gel after the completion of the radical polymerization is moved to the polyvalent ion container by sliding and dropping a bottom portion of the gel mold. Accordingly, the gel after the completion of the radical polymerization is immersed in the polyvalent ion solution, and a polymerization based on ion is started.

In the gel, completion of the polymerization based on ion is awaited. After the completion of the polymerization, the decompression chamber is returned to atmospheric pressure to collect the gel.

As described above, the gel for an acoustic coupler according to the present embodiment can be produced.

### EMBODIMENT

A method for producing a gel for an acoustic coupler according to an embodiment will be described.

### First Embodiment

<Method for Producing Gel according to First Embodiment>

Pressure in a decompression chamber was reduced to −20 mmHg by a vacuum pump, and a polyvalent ion solution, a raw material, and a polymerization agent introduced in a polyvalent ion server, a raw material server, and a polymerization agent server were degassed using an aspirator, separately.

As the polyvalent ion solution, a calcium oxalate aqueous solution having a concentration (hereinafter, a concentration (%) in the present embodiment is expressed as a percentage of w/v=weight (unit: g)/volume (unit: ml) unless otherwise specified) of 1% was used.

As the raw material, a liquid obtained by dissolving acrylamide, bisacrylamide, sodium alginate, and ammonium persulfate (APS) in distilled water at concentrations of 3.9%, 0.1%, 0.5%, and 0.1%, respectively was used.

In order to produce one gel, 100 ml of the raw material solution was used.

As the polymerization agent, tetramethylethylenediamine (TEMED) was used in an amount that a concentration expressed as a percentage of v/v=volume (unit: ml)/volume (unit: ml) was 0.05% with respect to a total amount of other components in the raw material of the gel.

Next, 500 ml of the polyvalent ion solution was transferred to the polyvalent ion container in the decompression chamber. In addition, 100 ml of the raw material and 0.1 ml of the polymerization agent were transferred to the gel mold and mixed by rotating the gel mold.

After the radical polymerization was completed for 20 minutes, a bottom portion of the gel mold was slid and dropped from the gel mold, and the solution obtained after completion of the radical polymerization was moved to the polyvalent ion container.

In this state, the solution was left for two days to perform the polymerization by ion.

Then, pressure of the decompression chamber was returned to atmospheric pressure to collect gel.

### COMPARATIVE EXAMPLE

As a comparative example, a gel was similarly produced in air without reducing pressure of the chamber.

### <Evaluation>

### (Breaking Strain)

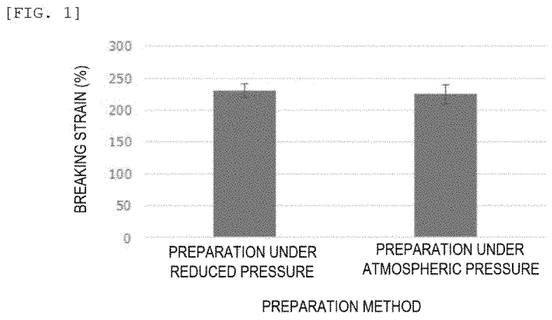

Breaking strain (degree of deformation) was measured for the gel produced according to the first embodiment and the gel produced according to the comparative example. A result thereof is shown in FIG. 1. The breaking strain was measured by elongating the gel by a tensile tester, measuring strain when the gel breaks, and calculating a value obtained by dividing the strain by a cross-sectional area of a sample of the gel.

The breaking strain was measured using five samples of the gel produced according to the first embodiment and five samples of the gel produced according to the comparative example. As a result, the breaking strain of the gel produced under reduced pressure according to the embodiment was 231±10.6%, and the breaking strain of the gel produced under the atmospheric pressure according to the comparative example was 225±14.6%.

### (Sound Velocity)

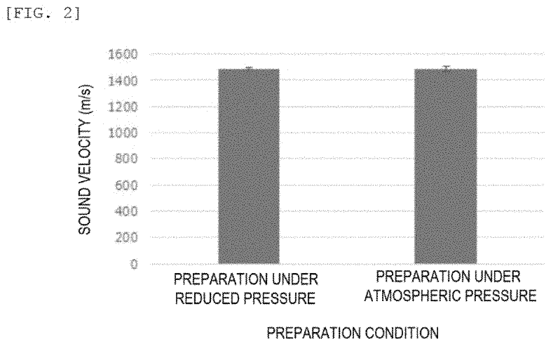

A sound velocity, which is an acoustic characteristic, was measured for the gel produced according to the first embodiment and the gel produced according to the comparative example. A result thereof is shown in FIG. 2. The sound velocity was measured by measuring an ultrasonic wave of 3.5 MHz by a pulsar receiver method at a temperature of 20° C.

As a result of measurement using five samples of the gel produced according to the first embodiment and five samples of the gel produced according to the comparative example, the sound velocity of the gel produced under the reduced pressure according to the embodiment was 1488±7.8 m/s, and the sound velocity of the gel produced under the atmospheric pressure was 1487±18.3 m/s.

### (Attenuation Rate)

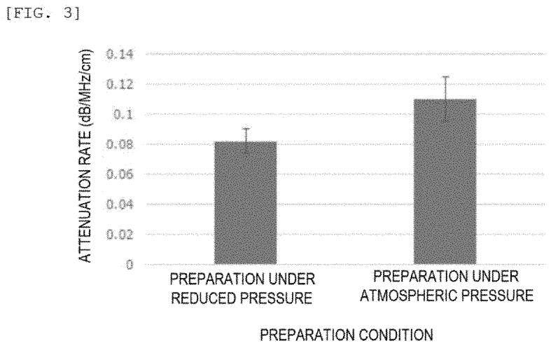

An attenuation rate, which is an acoustic characteristic, was measured for the gel according to the first embodiment and the gel according to the comparative example. A result thereof is shown in FIG. 3. The attenuation rate was measured by measuring an ultrasonic wave of 3.5 MHz by a pulsar receiver method at a temperature of 20° C. and then converting a unit into a unit of dB/MHz/cm.

As a result of measurement using five samples of the gel produced according to the first embodiment and five samples of the gel produced according to the comparative example, the attenuation rate of the gel produced under the reduced pressure according to the embodiment is 0.082±0.085 m/s, the attenuation rate of the gel produced under the atmospheric pressure according to the comparative example is 0.11±0.015 m/s, and the attenuation rate of the sample subjected to a reduced pressure adjustment is significantly smaller.

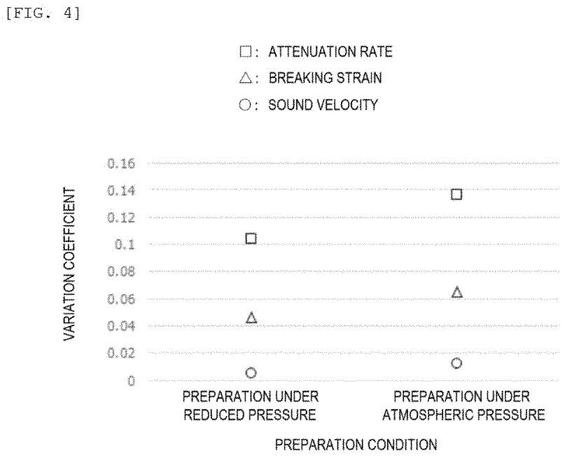

FIG. 4 shows results of FIGS. 1 to 3 as variation coefficients (standard deviation/average value). It was found that the variation coefficient of the sound velocity is the smallest and the variation coefficient of the attenuation rate is the largest. In particular, the variation coefficient of the attenuation rate of the gel produced under the atmospheric pressure according to the comparative example is slightly less than 14%, which is the same level as a sensitivity variation (substantially 20%) between elements of a probe of an ultrasonic diagnostic apparatus. It was found that, in order to perform quantitative ultrasonic measurement, it is desirable to use the gel produced under the reduced pressure according to the first embodiment.

### Second Embodiment

As a second embodiment, a gel was produced in the same manner as in the first embodiment except that a concentration of acrylamide in a raw material solution is changed between 3% and 6%. At this time, a concentration of bisacrylamide is adjusted to be 1/39 of the concentration of the acrylamide. Further, the row material solution was prepared in a manner that a concentration of sodium alginate is changed by 10% between 0 to 100% with respect to a total concentration of the acrylamide and the bisacrylamide. Further, according to a change in a raw material amount, an amount of TEMED was adjusted to 1/500 of the raw material amount and an amount of APS was adjusted to 1/1000 of the raw material amount.

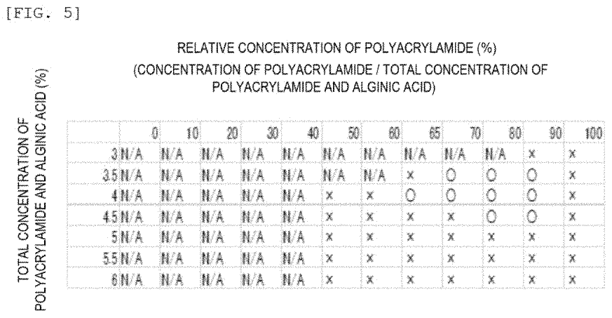

As a result, gels within a concentration range in which “◯” or “X” in a table shown in FIG. 5 was described was produced.

### (Mechanical Strength Evaluation)

Mechanical strength was tested for the gels produced according to the second embodiment. A result thereof is shown in FIG. 5.

As a test method, the gel (size: 50=×50=×15=) produced according to the second embodiment is fixed on a flat measurement surface such that a surface with the size of 15 mm is on a top. A stainless steel rod with a diameter of 30 mm is mounted on the top of the gel instead of an ultrasonic probe, and an operation of moving the stainless steel rod at a speed of once every two seconds such that a thickness of the gel becomes 10 mm is repeated 100 times. Thereafter, it is optically confirmed whether or not there is a crack in the gel.

As a result, a gel without crack is indicated by ◯, and a gel with crack is indicated by X. As shown in FIG. 5, a range where, in the gel, a total concentration of the polyacrylamide and the alginic acid is more than 3% and less than 5% and a relative concentration of the polyacrylamide to the total concentration of the polyacrylamide and the alginic acid is more than 60% and less than 100% is a range where there is no crack. Specifically, there is no crack in samples in which the relative concentration of the polyacrylamide and the total concentration of the polyacrylamide and the alginic acid which are indicated by marks ◯ in FIG. 5 are combined.

### REFERENCE SIGN LIST

1 . . . decompression chamber
5 . . . gel mold
9 . . . polyvalent ion solution container

1. A gel for an acoustic coupler to be disposed between a subject and a probe that transmits an ultrasonic wave, the gel for an acoustic coupler comprising:
polyacrylamide having a matrix structure; and
alginic acid, wherein
the alginic acid is retained in a matrix of the matrix structure of the polyacrylamide.

2. The gel for an acoustic coupler according to claim 1, wherein
the alginic acid retained in the matrix is crosslinked via an ion to form matrix-shaped alginic acid.

3. The gel for an acoustic coupler according to claim 1, wherein
a total concentration of the polyacrylamide and the alginic acid in the gel (hereinafter, a concentration (%) in the gel is expressed as a percentage of weight (unit: g)/volume (unit: ml) of the gel) is more than 3% and less than 5%.

4. The gel for an acoustic coupler according to claim 3, wherein
the total concentration of the polyacrylamide and the alginic acid in the gel is 3.5% or more and 4.5% or less.

5. The gel for an acoustic coupler according to claim 3, wherein
a relative concentration of the polyacrylamide to the total concentration of the polyacrylamide and the alginic acid in the gel is more than 60% and less than 100%.

6. The gel for an acoustic coupler according to claim 5, wherein
the relative concentration of the polyacrylamide to the total concentration of the polyacrylamide and the alginic acid in the gel is 65% or more and 90% or less.

7. The gel for an acoustic coupler according to claim 1, wherein
breaking strain is 200% or more, deviation of a sound velocity from a sound velocity of water is 5% or less, and an ultrasonic attenuation rate is 0.1 dB/MHz/cm or less.

8. A method for producing a gel for an acoustic coupler comprising:
a step of mixing a plurality of kinds of polymers having different polymerization systems or polymer raw materials;
a first gelling step of polymerizing or crosslinking a first kind of polymer or polymer raw material among the plurality of kinds of polymers or the polymer raw materials; and
a second gelling step of polymerizing or crosslinking a second kind of polymer or polymer raw material among the plurality of kinds of polymers or the polymer raw materials, wherein
all the steps are performed under reduced pressure.

9. The method for producing a gel for an acoustic coupler according to claim 8, wherein
one of the first gelling step and the second gelling step is a step of polymerizing with a radical generator, and the other of the first gelling step and the second gelling step is a step of crosslinking via a polyvalent ion.

10. The method for producing a gel for an acoustic coupler according to claim 9, wherein
the step of polymerizing with the radical generator is a step of forming polyacrylamide.

11. The method for producing a gel for an acoustic coupler according to claim 9, wherein
the step of crosslinking via the polyvalent ion is a step of crosslinking alginic acid via a polyvalent ion.

12. The method for producing a gel for an acoustic coupler according to claim 8, wherein
the mixing step is a step of mixing a raw material of polyacrylamide and a raw material containing alginic acid, and a total concentration of the raw material of the polyacrylamide and the alginic acid in the raw material after mixing (hereinafter, a concentration (%) in the raw material after mixing is expressed as a percentage of weight (unit: g)/volume (unit: ml) of the raw material after mixing) is more than 3% and less than 5%.

13. The method for producing a gel for an acoustic coupler according to claim 12, wherein
the total concentration of the raw material of the polyacrylamide and the alginic acid in the mixing step is 3.5% or more and 4.5% or less.

14. The method for producing a gel for an acoustic coupler according to claim 8, wherein
the mixing step is a step of mixing a raw material of polyacrylamide and a raw material containing alginic acid, and a relative concentration of the raw material of the polyacrylamide to a total concentration of the raw material of the polyacrylamide and the alginic acid in the raw material after mixing is more than 60% and less than 100%.

15. The method for producing a gel for an acoustic coupler according to claim 14, wherein
the relative concentration of the raw material of the polyacrylamide to the total concentration of the raw material of the polyacrylamide and the alginic acid in the mixing step is 65% or more and 90% or less.

16. An ultrasonic imaging method, comprising:
in a state where the acoustic coupler according to claim 1 is disposed between a subject and a probe that transmits an ultrasonic wave, irradiating an inside of the subject with an ultrasonic wave by causing the probe to transmit the ultrasonic wave and causing the ultrasonic wave to pass through the acoustic coupler;
receiving an ultrasonic wave from the subject toward the probe due to the ultrasonic wave irradiation by causing the ultrasonic wave to pass through the acoustic coupler and reach the probe; and
generating an ultrasonic image using the ultrasonic signal received by the probe.
